(19) United States
(12) Patent Application Publication
(10) Pub. No.: US 2016/0263386 A1
(43) Pub. Date: Sep. 15, 2016

(54) DEEP BRAIN STIMULATION CLINICAL EFFECTS MAP WITH VISUAL INDICATORS FOR PATIENT AMPLITUDE LIMITS

(71) Applicant: Boston Scientific Neuromodulation Corporation, Valencia, CA (US)

(72) Inventor: Dennis Allen Vansickle, Lancaster, CA (US)

(21) Appl. No.: 15/064,189

(22) Filed: Mar. 8, 2016

Related U.S. Application Data
(60) Provisional application No. 62/130,037, filed on Mar. 9, 2015.

(51) Int. Cl. A61N 1/372; A61N 1/36; A61N 1/05

(52) U.S. Cl. CPC A61N 1/37247; A61N 1/0551; A61N 1/0534; A61N 1/36146

(57) ABSTRACT

An apparatus comprises a port configured to receive indications of efficacy of electrical neurostimulation in treating a condition of a subject at different energy stimulation levels, a display, and a control circuit. The control circuit is configured to: initiate delivery of neurostimulation therapy using a plurality of implantable electrodes; present using the display a mapping of indications of neurostimulation efficacy with electrodes of the plurality of implantable electrodes used to provide the neurostimulation, and present with the mapping an indication of a recommended maximum energy amplitude setting for the therapy and an indication of a recommended minimum energy amplitude setting of the therapy.

### CLAIM OF PRIORITY

This application claims the benefit of priority under 35 U.S.C. §119(e) of U.S. Provisional Patent Application Ser. No. 62/130,037, filed on Mar. 9, 2015, which is herein incorporated by reference in its entirety.

### BACKGROUND

Neurostimulation, also referred to as neuromodulation, has been proposed as a therapy for a number of conditions. Some examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been developed to deliver this type of therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site of the nervous system. An external programming device can be used to program the implantable neurostimulator with stimulation parameters controlling the delivery of the neurostimulation energy.

Neurostimulation energy can be delivered in the form of electrical neurostimulation pulses. The energy delivery may be controlled using stimulation parameters that specify spatial (where to stimulate), temporal (when to stimulate), and informational (patterns of pulses directing the nervous system to respond as desired) aspects of a pattern of neurostimulation pulses. The human nervous systems use neural signals having much more sophisticated patterns to communicate various types of information, including sensations of pain, pressure, temperature, etc. The nervous systems may interpret an artificial stimulation with a simple pattern of stimuli as an unnatural phenomenon, and respond with an unintended and undesirable sensation and/or movement. Also, as the condition of the patient deteriorates, stimuli that in the past provided effective therapy to the patient may subsequently cause unintended and undesirable sensation and/or movement. Customization of device-based therapy to a patient may require periodic monitoring and updating. This can make programming therapy for a patient a challenging task.

### Overview

It is desirable for neurostimulation devices to provide the most effective therapy to a patient. Some doctors want to give their patient's some control over their therapy. However, a patient may not be sophisticated in the use and programming of the neurostimulation device. The present subject matter relates to improving therapy provided by neurostimulation devices.

An apparatus example of the present subject matter includes a port, a display, and a control circuit. The port can be configured to receive indications of efficacy of electrical neurostimulation in treating a condition of a subject at different energy stimulation levels. The control circuit can be configured to initiate delivery of neurostimulation therapy using a plurality of implantable electrodes, present using the display a mapping of indications of neurostimulation efficacy with electrodes of the plurality of implantable electrodes used to provide the neurostimulation, and present with the mapping an indication of a recommended maximum energy amplitude setting for the therapy and an indication of a recommended minimum energy amplitude setting of the therapy.

This section is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of an example of a neurostimulator device.

FIG. 2 illustrates an implantable pulse generator and a neurostimulation lead arranged to provide electrical deep brain stimulation.

FIG. 3 illustrates portions of a neurostimulation system.

FIG. 4 shows a block diagram of portions of an example of an external medical device.

FIG. 5 shows an example of a display that includes a therapy response map.

FIG. 6 shows another example of a display that includes a therapy response map.

FIG. 7 shows a block diagram of portions of an example of a medical device system.

FIG. 8 shows a flow diagram of a method of controlling operation of a neurostimulator device.

### DETAILED DESCRIPTION

This document discusses devices and methods for generating and delivering pacing therapy and neural stimulation therapy. Specifically, devices and methods for providing both pacing therapy and neural stimulation therapy via the same circuit are described.

A programmer for an implantable pulse generator (IPG) may include one or more of the features, structures, methods, or combinations thereof described herein.

For example, a programmer for an electrical neurostimulator may be implemented to include one or more of the advantageous features or processes described below. It is intended that such a device need not include all of the features described herein, but may be implemented to include selected features that provide for unique structures or functionality. Such a device may be implemented to provide a variety of therapeutic or diagnostic functions.

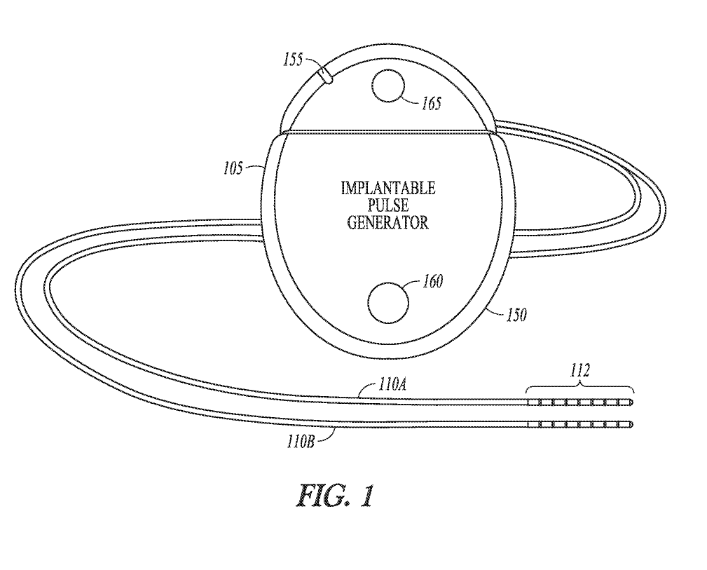

FIG. 1 illustrates an example of a neurostimulator device. The device includes an IPG 105 that can be coupled to one or more implantable neurostimulation leads 110A, 110B at a proximal end of the leads. The distal end includes electrical contacts or electrodes for contacting a tissue site targeted for electrical neurostimulation. In the example of FIG. 1, each lead includes eight electrodes 112. The number of electrodes shown in FIG. 1 is only an example and other arrangements are possible. In certain variations, the electrodes are ring electrodes. The implantable electrodes and leads may be configured by shape and size to provide electrical neurostimulation energy to a nerve cell target included in the subject's brain, or configured to provide electrical neurostimulation energy to a nerve cell target included in the subject's spinal cord.

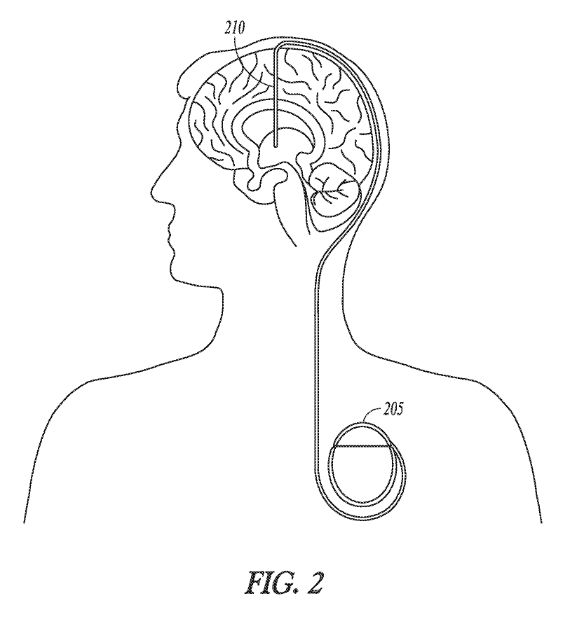

FIG. 2 illustrates an IPG 205 and a neurostimulation lead 210 arranged to provide DBS to a patient. In the example, the stimulation target is nerve cell tissue in a subdivision of the thalamus. Other examples of nerve cell targets of DBS include nerve cell tissue of the globus pallidus (GPi), the subthalamic nucleus (STN), and the pedunculopontine nucleus (PPN).

Returning to FIG. 1, the IPG 105 can include a hermetically-sealed IPG housing or can 150 to house the battery and the electronic circuitry of the IPG 105. The IPG 105 can include an electrode 160 formed on the IPG can 150. The IPG 105 can include an IPG header 155 for coupling the proximal ends of the leads. The IPG header 155 may optionally also include an electrode 165. Neurostimulation energy can be delivered in a unipolar mode using the electrode 160 or electrode 165 and one or more electrodes of a lead. Neurostimulation energy can be delivered in a bipolar mode using a pair of electrodes of a lead. Neurostimulation energy can be delivered in an extended bipolar mode using only one electrode of a lead (e.g., only one electrode of lead 110A) and one electrode of a different lead (e.g., only one electrode of lead 10B). Neurostimulation energy can be delivered in a monopolar mode using only one electrode of a lead without a second electrode.

The electronic circuitry of the IPG 105 can include a control circuit that controls delivery of the neurostimulation energy. The control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

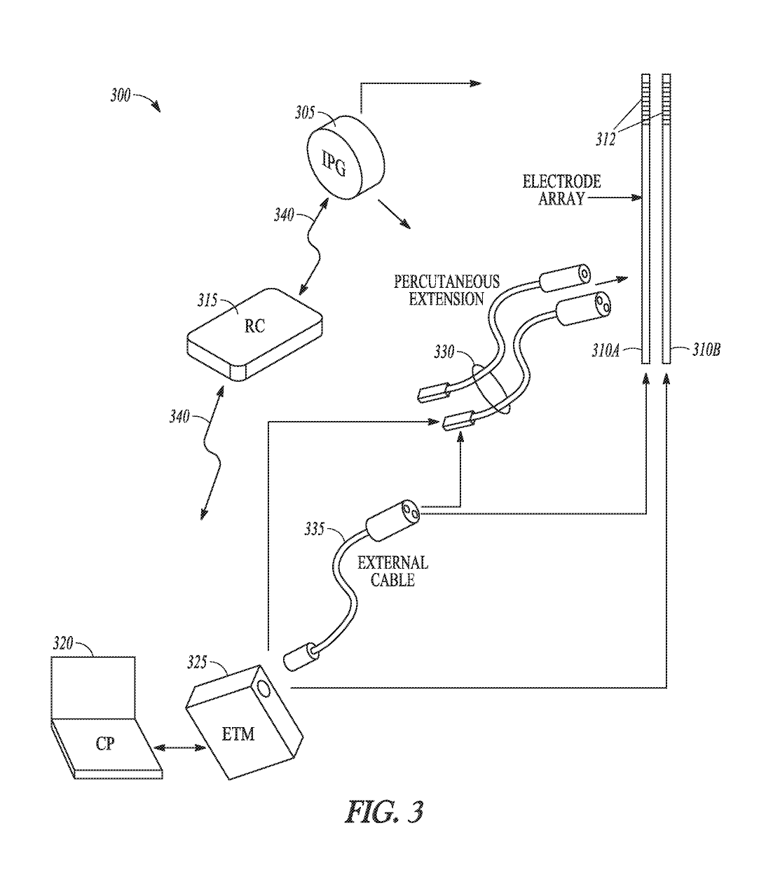

FIG. 3 illustrates portions of a neurostimulation system 300. The system includes an IPG 305, implantable neurostimulation leads 310A and 310B that include electrodes 312, an external remote controller (RC) 315, a clinician's programmer (CP) 320, and an external trial modulator (ETM) 325. The IPG 305 may be electrically coupled to the leads directly or through percutaneous extension leads 330.

The ETM 325 may be standalone or incorporated into the CP 320. The ETM 325 may also be electrically connectable to the neurostimulation leads 310A, 310B via one or both of the percutaneous extension leads 330 and external cable 335 to the neurostimulation leads 310A, 310B. The ETM 325 may have similar pulse generation circuitry as the IPG 305 to deliver neurostimulation energy according to specified modulation parameters as described previously. The ETM 325 is an external device that is typically used as a preliminary stimulator after the neurostimulation leads 310A, 310B have been implanted and used prior to stimulation with the IPG 305 to test the responsiveness of the stimulation that is to be provided. Because the ETM 325 is external it may be more easily configurable than the IPG 305.

The CP 320 can configure the neurostimulation provided by the ETM 325. If the ETM 325 is not integrated into the CP 320, the CP 320 may communicate with the ETM 325 using a wired connection (e.g., over a USB link) or by wireless telemetry using a wireless communications link 340. The CP 320 also communicates with the IPG 305 using a wireless communications link 340.

An example of wireless telemetry is based on inductive coupling between two closely-placed coils using the mutual inductance between these coils. This type of telemetry is referred to as inductive telemetry or near-field telemetry because the coils must typically be closely situated for obtaining inductively coupled communication.

The IPG 305 can include the first coil and a communication circuit hermetically-sealed LPG housing. The CP 320 includes the second coil in a wand that is electrically connected to the CP 320. Another example of wireless telemetry includes a radio frequency (RF) telemetry link or a far-field telemetry link. A far-field, also referred to as the Fraunhofer zone, refers to the zone in which a component of an electromagnetic field produced by the transmitting electromagnetic radiation source decays substantially proportionally to 1/r, where r is the distance between an observation point and the radiation source. Accordingly, far-field refers to the zone outside the boundary of r=λ/2π, where λ is the wavelength of the transmitted electromagnetic energy. In one example, a communication range of an RF telemetry link is at least two meters but can be as long as allowed by the particular communication technology. An RF antenna can be included in the header of the IPG 305 and in the housing of the CP 320; eliminating the need for a wand.

The CP 320 can be used to set modulation parameters for the neurostimulation after the IPG 305 has been implanted. This allows the neurostimulation to be tuned if the requirements for the neurostimulation change after implantation. The CP 320 may also upload information from the IPG 305.

The RC 315 also communicates with the IPG 305 using a wireless link 340. The RC 315 may be a communication device given to the patient or caregiver. The RC 315 may have reduced programming capability compared to the CP 320. This allows the patient to alter the neurostimulation therapy but does not allow the patient full control over the therapy. For example, the patient may be able to increase the amplitude of neurostimulation pulses or change the time that a preprogrammed stimulation pulse train is applied. The RC 315 may be programmed by the CP 320. The CP 320 may communicate with the RC 315 using a wired or wireless communications link. In some variations, the CP 320 is able to program the RC 315 when remotely located from the RC 315.

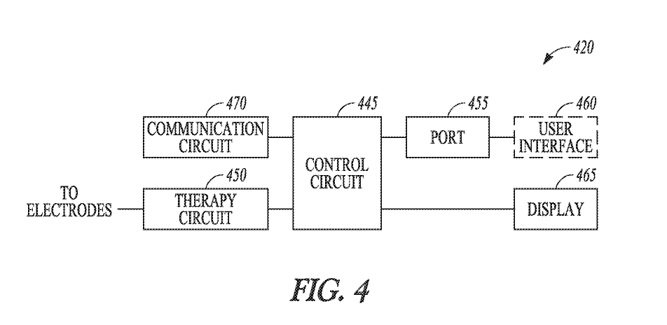

FIG. 4 shows a block diagram of portions of an example of an external medical device 420. An example of the external medical device is the CP 320 of the example of FIG. 3. The external medical device includes a control circuit 445 that initiates delivery of neurostimulation therapy using implantable electrodes. In some examples, the neurostimulation therapy is delivered using a therapy circuit 450. The therapy circuit 450 can be included in an ETM that is coupled to or integral to the external medical device. The implantable electrodes may be included in leads such as the implantable neurostimulation leads 110A, 110B in the example of FIG. 1.

In some examples, the control circuit 445 can specify energy stimulation levels on each of the electrodes independently. The example of FIG. 1 shows eight electrodes on each of the leads. The energy stimulation levels may differ in one or both of stimulation pulse amplitude and stimulation pulse width. An energy stimulation pulse may be a current pulse (e.g., measured in milliamps) or voltage pulse.

The external medical device includes a port 455 and a display 465. The port 455 receives indications of efficacy of electrical neurostimulation in treating a condition of a patient or subject at the different energy stimulation levels. In certain examples, the port 455 includes a communication port or “COMM Port” for communication with a separate device to receive information regarding therapy efficacy from the separate device. In certain examples, the port 455 is electrically coupled to a user interface 460 to receive input from a user. The user interface 460 may include one or more of the display 465, a mouse, a keyboard, and a touch sensitive or multi-touch sensitive display screen. Using the display 465, the control circuit 445 presents a mapping of indications of neurostimulation efficacy with electrodes of the plurality of implantable electrodes used to provide the neurostimulation.

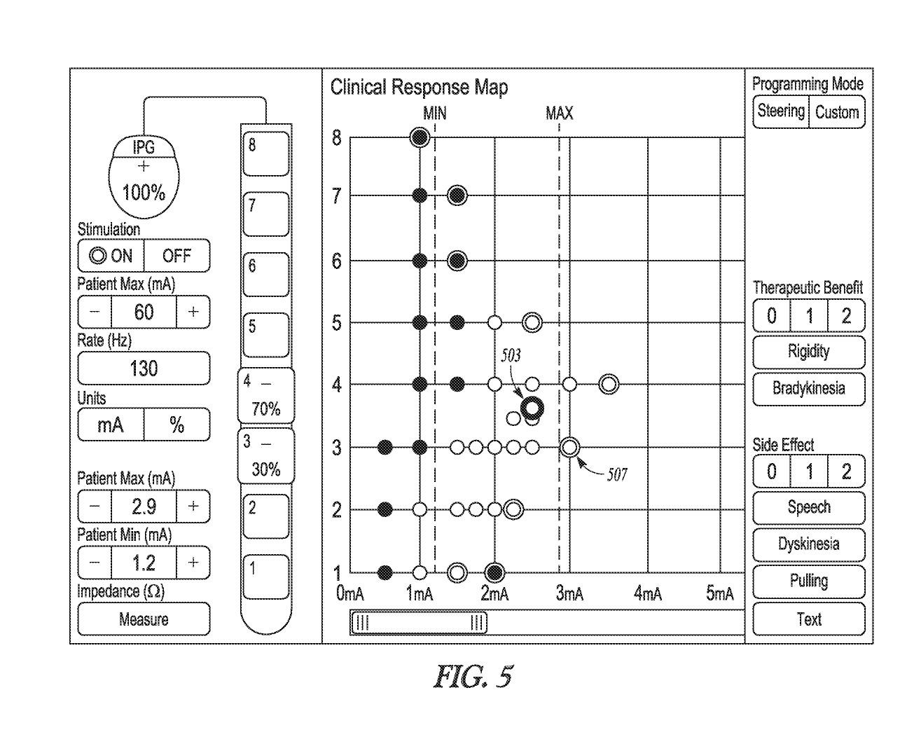

FIG. 5 shows an example of a display that includes a therapy response map. The vertical axis includes the electrodes used to provide the neurostimulation and the horizontal axis includes the amplitude of the neurostimulation. In this example, the neurostimulation provides pulses of constant current amplitude or quasi-constant current amplitude. The display in the example maps the efficacy of eight electrodes (e.g., the eight electrodes of a neurostimulation lead 110A or 110B of FIG. 1) to the amplitude of the neurostimulation as the amplitude is varied. The neurostimulation energy may be applied between the selected electrode and a common electrode or the neurostimulation may be applied in a monopolar mode using only the selected electrode.

Trials of the neurostimulation include an electrode and stimulation amplitude, and the trials correspond to circles on the display. To indicate efficacy of the neurostimulation, the circles are darker where there was no perceived benefit to the patient and lighter where stimulation was perceived to be more effective. In certain variations, the circles vary in color to indicate efficacy.

In the example of FIG. 5, the display is included in a user interface. A user such as a clinician may use the interface to input gradations of the perceived benefit on a scale from no perceived benefit to a strong perceived benefit. Most of the electrodes indicate an increase in benefit as the amplitude of the stimulation is increased. Electrode 1 however shows a case where increasing the amplitude did not improve the efficacy of the neurostimulation.

In certain examples, the neurostimulation can be applied using multiple electrodes. The display example of FIG. 5 shows a selection where seventy percent (70%) of the stimulation is provided using electrode 4 and 30% of the stimulation is provided using electrode 3. For electrical stimulation electrode combinations, the efficacy of the stimulation is represented by circles positioned between the electrodes used, such as the circles between electrodes 3 and 4 for example. The large ring 503 around a neurostimulation circle indicates the current neurostimulation programmed.

Included with the response mapping, the control circuit 445 presents an indication of the recommended maximum energy amplitude setting for the therapy and an indication of the recommended minimum energy amplitude setting of the therapy. In the example of FIG. 5 the indications include vertical bars labeled “MAX” and “MIN.” In addition to the graphical indication of the vertical bars, the control circuit 445 may also display the numerical values corresponding to the vertical bars; shown as 2.9 milliamps (mA) and 1.2 mA on the left side of the display.

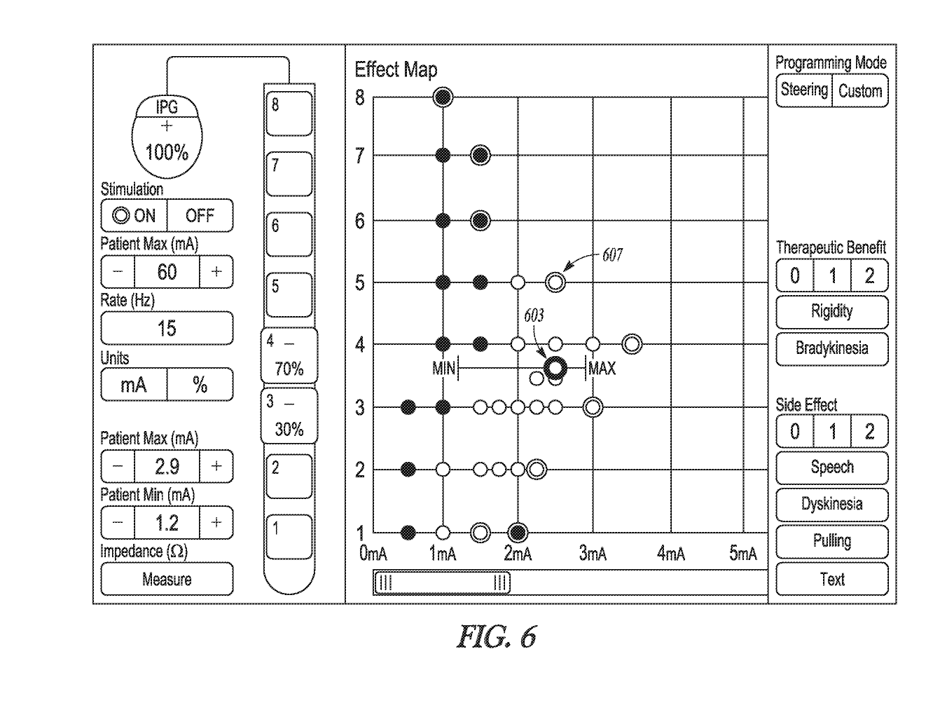

FIG. 6 shows another example of a display that includes a therapy response map. The indications of maximum energy amplitude setting and the minimum amplitude are included in the horizontal bar shown in FIG. 6. In certain examples, the horizontal bar is displayed in relation to the current neurostimulation programmed shown by the large ring 603 around the neurostimulation circle. In certain examples, the indications of maximum and minimum are configurable between the vertical option of FIG. 5 and the horizontal option of FIG. 6. In certain examples, the graphical indication of the maximum and minimum can be made visible or not visible.

Returning to FIG. 4, in some examples the external medical device includes a communication circuit 470 that communicates information to a separate remote control device such as the RC 315 in FIG. 3. The remote control device can be used to adjust electrical stimulation amplitude settings of an IPG. The communication circuit 470 communicates the values of the maximum and minimum energy amplitude settings to the remote control device and the remote control device uses the values as programmable limits for the electrical stimulation amplitude. If the remote control device is used by the patient, the patient is allowed to adjust the amplitude of the electrical stimulation over a range limited by the values communicated to the remote control device. This provides the patient some control of the therapy, but the patient is prevented from deviating too far from the therapy preferred by the clinician or physician.

In some examples, the recommended maximum and minimum energy amplitude settings are determined by the control circuit 445. As shown in the example of FIGS. 5 and 6, the control circuit 445 includes the energy stimulation levels provided to the electrodes in the display of the mapping of the indications of neurostimulation efficacy. The control circuit 445 may determine the maximum and minimum energy amplitude settings as recommended energy amplitude settings according to a range of energy amplitudes that are indicated to provide effective therapy to the patient. For instance, the control circuit 445 may identify the minimum setting corresponding to a specified minimum efficacy threshold. The maximum setting may be identified as the lowest maximum amplitude that achieves a second specified efficacy threshold. The values of the recommended settings may be communicated to the remote control device after receiving a confirmation of the settings via the user interface 460.

In some examples, the external medical device receives the maximum energy amplitude setting for the therapy and the minimum energy amplitude setting for the therapy via the user interface 460. For instance, a user may review the results of the electrostimulation trials present in the response mapping and set or adjust the vertical bars in FIG. 5 using the user interface. The control circuit 445 communicated the values of the settings to the remote control device.

According to some examples, the port 455 receives indications of side effects of the provided electrical neurostimulation. If the neurostimulation includes DBS, the side effects can be muscular and affect a patient's gait or cause dyskinesia, or the side effects can be neural and affect a patient's speech or vision. The control circuit 445 presents the side effects mapped to the neurostimulation efficacy and the individual electrodes used to provide the neurostimulation. In the example of FIGS. 5 and 6 side effects in the response mapping are indicated by highlighted circles around corresponding neurostimulation circles (e.g., circles 507 in FIG. 5 and circle 607 in FIG. 6). In certain examples, the side effects are observed by the clinician who inputs the side effects into the device through the user interface 460, such as shown to the right in FIGS. 5 and 6.

The control circuit 445 may determine recommended maximum and minimum energy amplitude settings according to the indications of the side effects and according to the effectiveness of the energy amplitudes of the therapy. For instance, the control circuit 446 may identify 1.2 mA as the minimum amplitude that provides effective therapy and identify 2.9 mA as the maximum amplitude that provides effective therapy without a certain side effect. In certain examples, the maximum may be set by the clinician to an amplitude value that provides effective therapy but results in acceptable side effects. With a well populated response map, the clinician can see where the stimulation amplitude limits coincide with clinical effects collected during the stimulation trials. In some examples, a response map may be stored in device memory and retrieved by a user for display. A stored response map may be useful to provide a history for the patient and can aid in understanding the progression of the patient's disease. For instance, review of a previous response map may show that current side effects were not previously present.

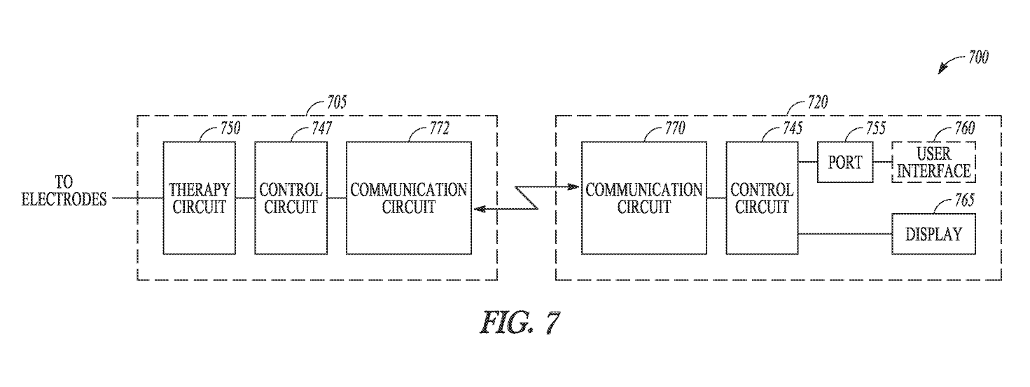

The examples described have included providing the electrical neurostimulation therapy for the therapy trials using an external device (e.g., an ETM). According to some examples, the therapy can be provided using an IPG. FIG. 7 shows a block diagram of portions of an example of a medical device system 700. The system includes an external medical device 720 and an IPG 705. The IPG 705 includes a therapy circuit 750 and a control circuit 747 to control delivery of neurostimulation energy by the therapy circuit 750. The IPG 705 provides neurostimulation to the patient using implantable electrodes and the IPG 705 and electrodes are implanted prior to delivery of neurostimulation energy to be mapped. The IPG 705 includes a communication circuit 772 to communicate information with the external medical device 720. The neurostimulation can be changed by wireless programming of modulation parameters in the IPG 705.

The external medical device 720 includes a port 755, a display 765, and a control circuit 745. As in the Example of FIG. 4, the port 755 may be electrically coupled to a COMM Port or to a user interface 760. The port 755 receives indications of efficacy of electrical neurostimulation in treating a condition of a patient or subject at different energy stimulation levels. The external medical device 720 also includes a communication circuit 770 to communicate information with the IPG 705.

Delivery of neurostimulation therapy by the IPG 705 is initiated by the control circuit 745 of the external medical device 720. Using the display 765, the control circuit 745 presents indications of neurostimulation efficacy mapped to the electrodes used to provide the neurostimulation. Included with the response mapping, the control circuit 745 presents an indication of a recommended maximum energy amplitude setting for the therapy and an indication of a recommended minimum energy amplitude setting of the therapy as shown in the examples of FIGS. 5 and 6.

The values of the maximum and minimum amplitude settings may then be communicated to a remote control device that uses the values as programmable limits for programming the electrical stimulation amplitude of the IPG 705.

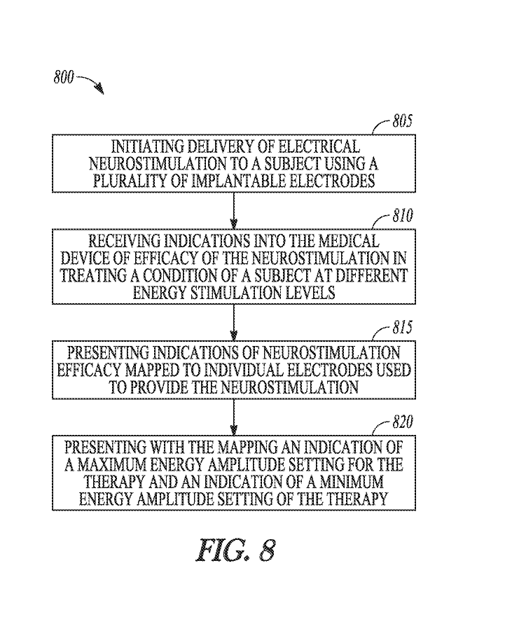

FIG. 8 shows a flow diagram of a method of controlling operation of a neurostimulation device. At 805, delivery of electrical neurostimulation to a subject is initiated using implantable electrodes. At 810, indications are received into the neurostimulation device of efficacy of the neurostimulation in treating a condition of a subject at different energy stimulation levels. The indications can be received into the neurostimulation from a user interface or from a separate device communicating with the neurostimulation device.

At 815, the neurostimulation device presents on a display indications of neurostimulation efficacy mapped to individual electrodes used to provide the neurostimulation. At 820, an indication of a recommended maximum energy amplitude setting for the therapy and an indication of a recommended minimum energy amplitude setting of the therapy are presented on the display with the mapping. Some examples of the response mapping were described previously herein in regard to FIGS. 5 and 6. The indications of the recommended maximum and minimum energy amplitude settings may be communicated to a separate remote control device that adjusts settings of an IPG that provides electrical stimulation therapy to the subject. The indications can be used to set programmable amplitude limits in the separate remote control device.

The devices, methods, and systems described herein are useful to customize device-based neurostimulation therapy to a patient. Device settings can give patients some control over their therapy even though a patient may be unsophisticated in device programming. Providing some limited control may increases the satisfaction of the patient with their device-based therapy.

### Additional Notes and Examples

Example 1 can include subject matter (such as an apparatus for electrically coupling to a plurality of electrodes) comprising a port configured to receive indications of efficacy of electrical neurostimulation in treating a condition of a subject at different energy stimulation levels, a display, and control circuit. The control circuit is optionally configured to initiate delivery of neurostimulation therapy using the plurality of implantable electrodes; present using the display a mapping of indications of neurostimulation efficacy with electrodes of the plurality of implantable electrodes used to provide the neurostimulation; and present with the mapping an indication of a recommended maximum energy amplitude setting for the therapy and an indication of a recommended minimum energy amplitude setting of the therapy.

In Example 2, the subject matter of Example 1 optionally includes a communication circuit configured to communicate the maximum and minimum energy amplitude settings to a separate remote control device that is configured to adjust electrical stimulation amplitude settings of an implantable pulse generator (IPG).

In Example 3, the subject matter of Example 1 or Example 2 optionally includes a control circuit configured to: include energy stimulation levels provided to the electrodes in the mapping of indications of neurostimulation efficacy, and determine the maximum and minimum energy amplitude settings as recommended energy amplitude settings according to a range of energy amplitudes that are indicated to provide effective therapy to the patient.

In Example 4, the subject matter of one or any combination of Examples 1-3 optionally includes a port configured to receive indications of side effects of the provided electrical neurostimulation, wherein the control circuit is configured to: present the side effects mapped to the neurostimulation efficacy and the individual electrodes used to provide the neurostimulation; and determine the maximum and minimum energy amplitude settings as recommended energy amplitude settings according to the indications of the side effects and according to energy amplitudes that are indicated to provide effective therapy to the patient.

In Example 5, the subject matter of one or any combination of Examples 1-4 optionally includes a user interface electrically coupled to the port and configured to receive the maximum energy amplitude setting for the therapy and the minimum energy amplitude setting for the therapy.

In Example 6, the subject matter of one or any combination of Examples 1-5 optionally includes the plurality of implantable electrodes. The implantable electrodes are optionally configured to provide neurostimulation energy to at least one of a nerve cell target included in the subject's spinal cord or a nerve cell target included in the subject's brain.

Example 7 can include subject matter (such as a method, a means for performing acts, or a device-readable medium including instructions that, when performed by the device, cause the device to perform acts), or can optionally be combined with the subject matter of one or any combination of Examples 1-6 to include such subject matter, comprising: initiating, using the medical device, receiving indications into the medical device of efficacy of electrical stimulation pre-delivered to a subject, the electrical stimulation having been delivered at different energy stimulation levels; presenting, with the medical device, indications of stimulation efficacy mapped to individual electrodes used to provide the stimulation; and presenting with the mapping an indication of a maximum energy amplitude setting for the therapy and an indication of a minimum energy amplitude setting of the therapy.

In Example 8, the subject matter of Example 7 optionally includes including communicating the indications of the recommended maximum energy amplitude setting and the minimum energy amplitude setting to a separate remote control device configured to adjust settings of an implantable pulse generator (IPG) that provides electrical stimulation pulses to the subject, wherein the indications set programmable amplitude limits in the separate remote control device.

In Example 9, the subject matter of Example 7 or Example 8 optionally includes determining energy stimulation levels provided to the individual electrodes; and presenting energy stimulation levels mapped to the individual electrodes and the stimulation efficacy.

In Example 10, the subject matter of Example 9 optionally includes determining, with the medical device, a recommended maximum energy amplitude setting and a recommended minimum energy amplitude setting according to a range of energy amplitudes that are determined to provide effective therapy to the patient; and presenting indications of the recommended maximum energy amplitude setting and the recommended minimum energy amplitude setting with the mapping.

In Example 11 the subject matter of one or any combination of Examples 7-10 optionally includes receiving indications of side effects of the provided electrical stimulation into the medical device, and presenting, with the medical device, the side effects mapped to the individual electrodes used to provide the stimulation and mapped to the stimulation efficacy.

In Example 12, the subject matter of Example 11 can optionally include determining a maximum energy amplitude determined using the indications of the side effects and determining a minimum energy amplitude setting using the indications of efficacy of stimulation, and wherein presenting the indications of a maximum energy amplitude setting and a minimum energy amplitude setting includes presenting indications of the recommended maximum and minimum energy amplitude settings.

In Example 13, the subject matter of Example 11 or Example 12 can optionally include receiving indications of the side effects via a user interface of the medical device.

In Example 14, the subject matter of one or any combination of Examples 7-13 optionally includes receiving indications of efficacy of the electrical stimulation in treating at least one of Spinal Cord Stimulation (SCS), Deep Brain Stimulation (DBS), Peripheral Nerve Stimulation (PNS), or Functional Electrical Stimulation (FES).

In Example 15, the subject matter of one or any combination of Examples 7-15 optionally includes receiving an adjustment to one or both of the maximum and energy amplitude setting and the minimum energy amplitude setting via a user interface of the medical device; and communicating values of the maximum energy amplitude setting and the minimum energy amplitude setting to a separate remote control device configured to adjust energy amplitude settings of an implantable pulse generator (IPG) that provides electrical stimulation therapy to the subject, wherein the indications set programmable amplitude limits in the separate remote control device.

Example 16 can include subject matter (such as an apparatus for electrically coupling to a plurality of electrodes), or can optionally be combined with the subject matter of one or any combination of Examples 1-15 to include such subject matter, comprising a port configured to receive indications of efficacy of electrical neurostimulation in treating a condition of a subject at different energy stimulation levels, a display, and control circuit. The control circuit is optionally configured to initiate delivery of neurostimulation therapy using the plurality of implantable electrodes; present using the display a mapping of indications of neurostimulation efficacy with electrodes of the plurality of implantable electrodes used to provide the neurostimulation; and present with the mapping an indication of a recommended maximum energy amplitude setting for the therapy and an indication of a recommended minimum energy amplitude setting of the therapy.

In Example 17, the subject matter of Example 16 optionally includes a communication circuit configured to communicate the maximum and minimum energy amplitude settings to a separate remote control device that is configured to adjust electrical stimulation amplitude settings of an implantable pulse generator (IPG).

In Example 18, the subject matter of Example 16 or Example 17 optionally includes a control circuit configured to: include energy stimulation levels provided to the electrodes in the mapping of indications of neurostimulation efficacy; and determine the maximum and minimum energy amplitude settings as recommended energy amplitude settings according to a range of energy amplitudes that are indicated to provide effective therapy to the patient.

In Example 19, the subject matter of one or any combination of Examples 16-18 optionally includes a port configured to receive indications of side effects of the provided electrical neurostimulation, wherein the control circuit is configured to: present the side effects mapped to the neurostimulation efficacy and the individual electrodes used to provide the neurostimulation; and determine the maximum and minimum energy amplitude settings as recommended energy amplitude settings according to the indications of the side effects and according to energy amplitudes that are indicated to provide effective therapy to the patient.

In Example 20, the subject matter of one or any combination of Examples 16-19 optionally includes a user interface electrically coupled to the port and configured to receive the maximum energy amplitude setting for the therapy and the minimum energy amplitude setting for the therapy.

In Example 21, the subject matter of one or any combination of Examples 16-20 optionally includes the plurality of implantable electrodes. The implantable electrodes are optionally configured to provide neurostimulation energy to at least one of a nerve cell target included in the subject's spinal cord or a nerve cell target included in the subject's brain.

Example 22 can include subject matter (such as a method, a means for performing acts, or a device-readable medium including instructions that, when performed by the device, cause the device to perform acts), or can optionally be combined with the subject matter of one or any combination of Examples 1-21 to include such subject matter, comprising: initiating, using the medical device, delivery of electrical neurostimulation to a subject using a plurality of implantable electrodes; receiving indications into the medical device of efficacy of the neurostimulation in treating a condition of a subject at different energy stimulation levels; presenting, with the medical device, indications of neurostimulation efficacy mapped to individual electrodes used to provide the neurostimulation; and presenting with the mapping an indication of a maximum energy amplitude setting for the therapy and an indication of a minimum energy amplitude setting of the therapy.

In Example 23, the subject matter of Example 22 optionally includes communicating the indications of the recommended maximum energy amplitude setting and the minimum energy amplitude setting to a separate remote control device configured to adjust settings of an implantable pulse generator (IPG) that provides electrical stimulation therapy to the subject, wherein the indications set programmable amplitude limits in the separate remote control device.

In Example 24, the subject matter of Example 22 or Example 23 optionally includes determining energy stimulation levels provided to the individual electrodes; and presenting energy stimulation levels mapped to the individual electrodes and the neurostimulation efficacy.

In Example 25, the subject matter of Example 24 optionally includes determining, with the medical device, a recommended maximum energy amplitude setting and a recommended minimum energy amplitude setting according to a range of energy amplitudes that are determined to provide effective therapy to the patient; and presenting indications of the recommended maximum energy amplitude setting and the recommended minimum energy amplitude setting with the mapping.

In Example 26 the subject matter of one or any combination of Examples 22-25 optionally includes receiving indications of side effects of the provided electrical neurostimulation into the medical device; and presenting, with the medical device, the side effects mapped to the individual electrodes used to provide the neurostimulation and to the neurostimulation efficacy.

In Example 27, the subject matter of Example 26 can optionally include determining a maximum energy amplitude determined using the indications of the side effects and determining a minimum energy amplitude setting using the indications of efficacy of neurostimulation, and wherein presenting the indications of a maximum energy amplitude setting and a minimum energy amplitude setting includes presenting indications of the recommended maximum and minimum energy amplitude settings.

In Example 28, the subject matter of Example 26 or Example 27 can optionally include receiving indications of the side effects via a user interface of the medical device.

In Example 29, the subject matter of one or any combination of Examples 22-28 optionally includes receiving indications of efficacy of the electrical neurostimulation in treating at least one of Spinal Cord Stimulation (SCS), Deep Brain Stimulation (DBS), Peripheral Nerve Stimulation (PNS), or Functional Electrical Stimulation (FES).

In Example 30, the subject matter of one or any combination of Examples 22-29 optionally includes receiving an adjustment to one or both of the maximum and energy amplitude setting and the minimum energy amplitude setting via a user interface of the medical device; and communicating values of the maximum energy amplitude setting and the minimum energy amplitude setting to a separate remote control device configured to adjust energy amplitude settings of an implantable pulse generator (IPG) that provides electrical stimulation therapy to the subject, wherein the indications set programmable amplitude limits in the separate remote control device.

Example 31 can include subject matter (such as an apparatus) or can optionally be combined with the subject matter of one or any combination of Examples 1-30 to include such subject matter comprising: a port configured to receive indications of efficacy of electrical neurostimulation in treating a condition of a subject at different energy stimulation levels; a communication circuit configured to communicate information with an implantable pulse generator (IPG) configured to provide the electrical neurostimulation using a plurality of implantable electrodes; a display; and a control circuit configured to initiate delivery of neurostimulation therapy by the IPG; present indications using the display of neurostimulation efficacy mapped to electrodes of the plurality of implantable electrodes used to provide the neurostimulation; and present with the mapping an indication of a maximum energy amplitude setting for the therapy and an indication of a minimum energy amplitude setting of the therapy.

In Example 32, the subject matter of Example 31 can optionally include a communication circuit configured to communicate the indications of the maximum and minimum energy amplitude settings as programmable amplitude limits to a separate remote control device that is configured to adjust stimulation energy amplitude settings of the IPG.

In Example 33, the subject matter of Example 31 or Example 32 can optionally include a control circuit is configured to: include energy stimulation levels provided to the electrodes in the mapping of indications of neurostimulation efficacy; and determine the maximum and minimum energy amplitude settings as recommended energy amplitude settings according to a range of energy amplitudes that are indicated to provide effective therapy to the patient.

In Example 34, the subject matter of one or any combination of Examples 31-33 optionally includes a port configured to receive indications of side effects of the provided electrical neurostimulation, wherein the control circuit is configured to: present the side effects mapped to the neurostimulation efficacy and the individual electrodes used to provide the neurostimulation; and determine the maximum and minimum energy amplitude settings as recommended energy amplitude settings according to the indications of the side effects and according to energy amplitudes that are indicated to provide effective therapy to the patient.

In Example 35, the subject matter of one or any combination of Examples 31-34 can optionally include a user interface electrically coupled to the port and configured to receive the maximum energy amplitude setting for the therapy and the minimum energy amplitude setting for the therapy.

Example 36 can include, or can optionally be combined with any portion or combination of any portions of any one or more of Examples 1-35 to include, subject matter that can include means for performing any one or more of the functions of Examples 1-35, or a machine-readable medium including instructions that, when performed by a machine, cause the machine to perform any one or more of the functions of Examples 1-35.

These non-limiting examples can be combined in any permutation or combination.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown and described. However, the present inventors also contemplate examples in which only those elements shown and described are provided.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of“at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code may be tangibly stored on one or more volatile or non-volatile computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. An apparatus for electrical connection to a plurality of implantable electrodes, the apparatus comprising:
a port configured to receive indications of efficacy of electrical neurostimulation in treating a condition of a subject at different energy stimulation levels;
a display; and
a control circuit configured to: initiate delivery of neurostimulation therapy using the plurality of implantable electrodes; present using the display a mapping of indications of neurostimulation efficacy with electrodes of the plurality of implantable electrodes used to provide the neurostimulation; and present with the mapping an indication of a recommended maximum energy amplitude setting for the therapy and an indication of a recommended minimum energy amplitude setting of the therapy.

2. The apparatus of claim 1, including a communication circuit configured to communicate the maximum and minimum energy amplitude settings to a separate remote control device that is configured to adjust electrical stimulation amplitude settings of an implantable pulse generator (IPG).

3. The apparatus of claim 1, wherein the control circuit is configured to: include energy stimulation levels provided to the electrodes in the mapping of indications of neurostimulation efficacy; and determine the maximum and minimum energy amplitude settings as recommended energy amplitude settings according to a range of energy amplitudes that are indicated to provide effective therapy to the patient.

4. The apparatus of claim 1, wherein the port is configured to receive indications of side effects of the provided electrical neurostimulation, wherein the control circuit is configured to: present the side effects mapped to the neurostimulation efficacy and the individual electrodes used to provide the neurostimulation; and determine the maximum and minimum energy amplitude settings as recommended energy amplitude settings according to the indications of the side effects and according to energy amplitudes that are indicated to provide effective therapy to the patient.

5. The apparatus of claim 1, including a user interface electrically coupled to the port and configured to receive the maximum energy amplitude setting for the therapy and the minimum energy amplitude setting for the therapy.

6. The apparatus of claim 1, including the plurality of implantable electrodes, wherein the implantable electrodes are configured to provide neurostimulation energy to at least one of a nerve cell target included in the subject's spinal cord or a nerve cell target included in the subject's brain.

7. A method of controlling operation of a medical device, the method comprising:
initiating, using the medical device, delivery of electrical neurostimulation to a subject using a plurality of implantable electrodes;
receiving indications into the medical device of efficacy of neurostimulation in treating a condition of a subject at different energy stimulation levels;
presenting, with the medical device, indications of neurostimulation efficacy mapped to individual electrodes used to provide the neurostimulation; and
presenting with the mapping an indication of a maximum energy amplitude setting for the therapy and an indication of a minimum energy amplitude setting of the therapy.

8. The method of claim 7, including communicating the indications of the recommended maximum energy amplitude setting and the minimum energy amplitude setting to a separate remote control device configured to adjust settings of an implantable pulse generator (IPG) that provides electrical stimulation therapy to the subject, wherein the indications set programmable amplitude limits in the separate remote control device.

9. The method of claim 7, wherein presenting the indications of maximum and minimum energy amplitude includes:
determining energy stimulation levels provided to the individual electrodes; and
presenting energy stimulation levels mapped to the individual electrodes and the neurostimulation efficacy.

10. The method of claim 9, including:
determining, with the medical device, a recommended maximum energy amplitude setting and a recommended minimum energy amplitude setting according to a range of energy amplitudes that are determined to provide effective therapy to the patient; and
presenting indications of the recommended maximum energy amplitude setting and the recommended minimum energy amplitude setting with the mapping.

11. The method of claim 7, including:
receiving indications of side effects of the provided electrical neurostimulation into the medical device, and
presenting, with the medical device, the side effects mapped to the individual electrodes used to provide the neurostimulation and mapped to the neurostimulation efficacy.

12. The method of claim 11, including determining a maximum energy amplitude determined using the indications of the side effects and determining a minimum energy amplitude setting using the indications of efficacy of neurostimulation, and wherein presenting the indications of a maximum energy amplitude setting and a minimum energy amplitude setting includes presenting indications of the recommended maximum and minimum energy amplitude settings.

13. The method of claim 11, wherein receiving indications of side effects includes receiving indications of the side effects via a user interface of the medical device.

14. The method of claim 7, wherein receiving indications of efficacy into the device includes receiving indications of efficacy of the electrical neurostimulation in treating at least one of Spinal Cord Stimulation (SCS), Deep Brain Stimulation (DBS), Peripheral Nerve Stimulation (PNS), or Functional Electrical Stimulation (FES).

15. The method of claim 7, including receiving an adjustment to one or both of the maximum and energy amplitude setting and the minimum energy amplitude setting via a user interface of the medical device; and communicating values of the maximum energy amplitude setting and the minimum energy amplitude setting to a separate remote control device configured to adjust energy amplitude settings of an implantable pulse generator (IPG) that provides electrical stimulation therapy to the subject, wherein the indications set programmable amplitude limits in the separate remote control device.

16. An apparatus comprising:
a port configured to receive indications of efficacy of electrical neurostimulation in treating a condition of a subject at different energy stimulation levels;
a communication circuit configured to communicate information with an implantable pulse generator (IPG) configured to provide the electrical neurostimulation using a plurality of implantable electrodes;
a display; and
a control circuit configured to initiate delivery of neurostimulation therapy by the IPG; present indications using the display of neurostimulation efficacy mapped to electrodes of the plurality of implantable electrodes used to provide the neurostimulation; and present with the mapping an indication of a maximum energy amplitude setting for the therapy and an indication of a minimum energy amplitude setting of the therapy.

17. The apparatus of claim 16, wherein the communication circuit is configured to communicate the indications of the maximum and minimum energy amplitude settings as programmable amplitude limits to a separate remote control device that is configured to adjust stimulation energy amplitude settings of the IPG.

18. The apparatus of claim 16, wherein the control circuit is configured to: include energy stimulation levels provided to the electrodes in the mapping of indications of neurostimulation efficacy; and determine the maximum and minimum energy amplitude settings as recommended energy amplitude settings according to a range of energy amplitudes that are indicated to provide effective therapy to the patient.

19. The apparatus of claim 16, wherein the port is configured to receive indications of side effects of the provided electrical neurostimulation, wherein the control circuit is configured to: present the side effects mapped to the neurostimulation efficacy and the individual electrodes used to provide the neurostimulation; and determine the maximum and minimum energy amplitude settings as recommended energy amplitude settings according to the indications of the side effects and according to energy amplitudes that are indicated to provide effective therapy to the patient.

20. The apparatus of claim 16, including a user interface electrically coupled to the port and configured to receive the maximum energy amplitude setting for the therapy and the minimum energy amplitude setting for the therapy.
